(19) United States
(12) Patent Application Publication
(10) Pub. No.: US 2014/0053848 A1
(43) Pub. Date: Feb. 27, 2014

(54) PROTECTIVE DEVICE FOR IMPLANTED MEDICAL DEVICE

(71) Applicant: INSPIRED IDEAS, INC., Mesquite, TX (US)

(72) Inventor: David F. STRAND, Mesquite, TX (US)

(21) Appl. No.: 14/072,852

(22) Filed: Nov. 6, 2013

Related U.S. Application Data
(63) Continuation of application No. 13/163,793, filed on Jun. 20, 2011.

(51) Int. Cl. A61F 15/00

(52) U.S. Cl. CPC A61F 15/008
USPC 128/846

(57) ABSTRACT

A protective device for protecting a tender area proximate an implanted medical device, such as implanted venous access ports, stints, and shunts. In one embodiment the device has a cushion layer with an opening dimensioned to receive at least a portion of skin that protrudes outwardly due to an implanted medical device. For example, the cushion layer may be formed from a material configured to dampen a force applied to the protruding area. The device includes an adhesive layer for attaching the cushion layer to the skin. Typically, the cushion layer has a thickness that is greater than a distance that the protruding area extends outwardly due to the implanted medical device.

### RELATED APPLICATIONS

This is a continuation application of application Ser. No. 13/163,793 filed Jun. 20, 2011, which claims the benefit of U.S. Provisional Application No. 61/356,702, filed Jun. 21, 2010, the disclosures of which are hereby incorporated by reference in its entirety.

### TECHNICAL FIELD

This invention relates to a protective device for implanted medical devices; in particular, the invention relates to a device that provides cushioning to protect a tender area surrounding an implanted medical device that causes an area of skin to protrude outwardly, such as a venous access port.

### BACKGROUND

Many medical patients suffer discomfort caused by implanted medical devices, such as implanted venous access ports, stints, and shunts. For example, cancer patients who undergo chemotherapy and other types of infusion treatments will often choose to have a venous access port surgically implanted in their chest beneath the skin for use in such treatments.

Although the use of a venous access port reduces the mental and physical stress to the patient resulting from numerous IVs that would otherwise be required for the infusion treatments, there are downsides to such implanted medical devices. For example, a venous access port usually results in a tender area of skin that protrudes from the chest. Consequently, the protruding area is constantly irritated by other objects rubbing against it, such as seat belts, bra straps, and other articles of clothing. The constant irritation causes further discomfort to the patient.

Therefore, there is a need for a protective device that will reduce the irritation associated with an implanted medical device and, consequently, improve a patient's quality of life throughout the period of use of the implanted medical device.

### SUMMARY

According to one aspect, the invention provides a cushioned device that protects a tender area associated with an implanted medical device, such as a venous access port used in chemotherapy and other types of infusion treatments. As discussed above, these implanted devices cause a portion of skin to protrude outwardly, which can be a source of irritation to the user. The protective device includes a cushion layer that dampens force to the tender area to reduce irritation and an adhesive layer for attaching the cushion layer to the skin. In some embodiments, the cushion layer may have an opening dimensioned to receive at least a portion of a protruding area of skin. Typically, the cushion layer has a thickness that is greater than a distance that the protruding area extends outwardly due to the implanted medical device to reduce the chance of direct contact with the tender area.

In some exemplary embodiment, the cushion layer may be configured so the protruding area of skin extends into the opening when the adhesive layer is attached to the skin. In some cases, the cushion layer could be configured to transmit a force impacting the cushion layer to an area surrounding the protruding area. Typically, the cushion layer has a ring-like shape. Depending on the circumstances, the opening could be approximately concentric with respect to the cushion layer.

According to another aspect, the invention provides a method for protecting an implanted medical device. The method includes the step providing a foam blank with an adhesive adjacent a cushion layer and a protective film. An annular shape is cut into the foam blank to form a protective device with an opening dimensioned to receive an implanted medical device. The protective device may be removed from the foam blank and the protective film removed from the device. The device may then be adhered to skin surrounding an imbedded medical device. Typically, the cushion layer has a thickness that is greater than a distance that the skin protrudes due to the implanted medical device.

Additional features and advantages of the invention will become apparent to those skilled in the art upon consideration of the following detailed descriptions exemplifying the best mode of carrying out the invention as presently perceived.

### BRIEF DESCRIPTION OF THE DRAWINGS

The present disclosure will be described hereinafter with reference to the attached drawings which are given as non-limiting examples only, in which:

FIGS. 1 and 2 reflect a user removing the center section of the protective device from its proposed die-cut foam packaging and disposing of the same.

FIGS. 3 and 4 reflect a user separating the protective device from the proposed die-cut foam packaging.

FIG. 5 reflects removal of the protective film from the adhesive on the protective device by a user. Once the protective film is removed, the protective device is ready to be placed on the body around the protruding skin caused by an implanted medical device.

FIGS. 6 and 7 reflect the user adhering the protective device on the body around the protruding skin caused by an implanted medical device from both front and side views.

FIGS. 8 and 9 reflect the protective device adhered to the user from both front and side views.

FIG. 10 reflects the removal of the protective device by the user once the use is complete.

FIG. 11 reflects the disposal of the protective device by the user after removed from the body.

The components in the Figures are not necessarily to scale, emphasis instead being placed upon illustrating the principals of the invention. The exemplification set out herein illustrates embodiments of the invention, and such exemplification is not to be construed as limiting the scope of the invention in any matter.

### DISCLOSURE OF THE ILLUSTRATIVE EMBODIMENT

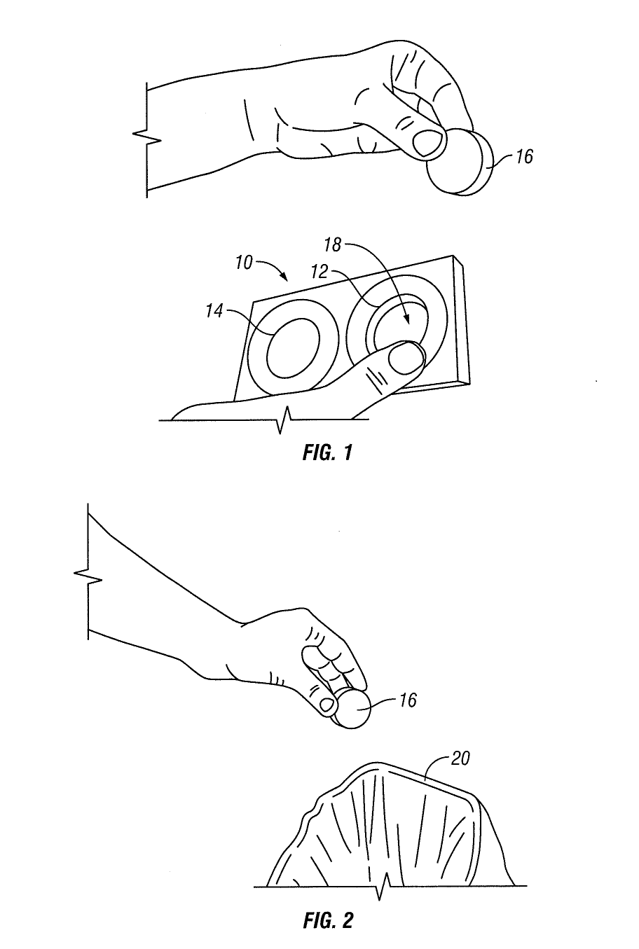

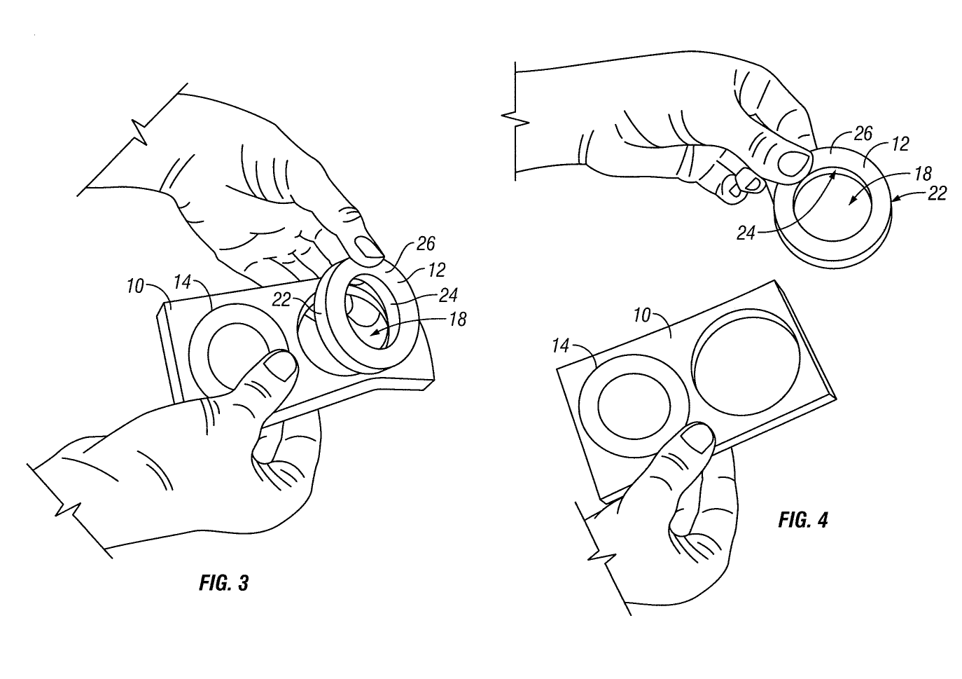

FIGS. 1 and 2 show an example embodiment of a protective device. The protective device may be shaped as a ring and may include and/or be applied with an adhesive for attaching the ring to the body. In some embodiments, the protective device may be formed from foam with a hole to create a substantially annular structure to surround an implanted medical device, such as a venous access port used for chemotherapy.

FIGS. 1 and 2 show a foam blank 10 with an example first protective device 12 and a second protective device 14 that have been formed in the foam blank, such as using a die cutter or laser. As shown in FIG. 1, a user may remove a center core 16 from the foam blank 10 prior to use. This creates an opening 18 in the protective device 12 that is dimensioned to surround the skin around the implanted medical device. FIG. 2 shows that the center core 16 may be discarded, such as in the trash 20.

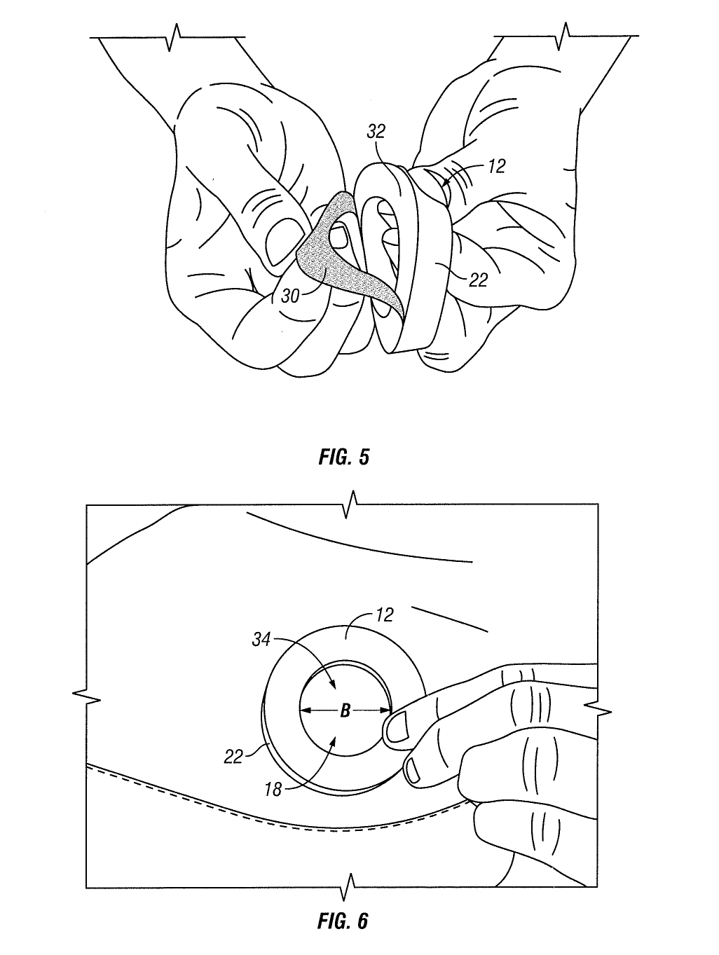

FIGS. 3 and 4 reflect a potential user separating the first protective device 12 from the foam blank 10. In this example, the center core 16 has been removed prior to separating the protective device 12 from the blank 10. However, embodiments are contemplated in which the center core 16 could be removed from the protective device 12 after the protective device 12 is separated from the blank 10. Although the protective device 12 shown in the figures has a circular shape and the opening 18 is circular, this is shown for example purposes only. Embodiments are contemplated in which the opening 18 could be circular, triangular, square, polygon or any other shape so long as the opening 18 is sufficiently dimensioned to surround the implanted medical device. In some embodiments, the protective device 12 could have a variety of shapes, such as circular, square, triangular, polygon or any other shape. In the example shown, the protective device 12 includes an outer wall 22 and an inner wall 24. As shown, the protective device 12 includes a front face 26 and a rear face 28.

FIG. 5 shows the removal of a protective film 30 from either the front face 26 or the rear face 28 of the protective device 12. The removal of the protective film 30 exposes an adhesive 32 on the protective device 12 for securing the protective device 12 to the skin of a user. Once the protective film 30 is removed, the protective device 12 is ready to be placed on the user's body to cushion the area surrounding the implanted medical device.

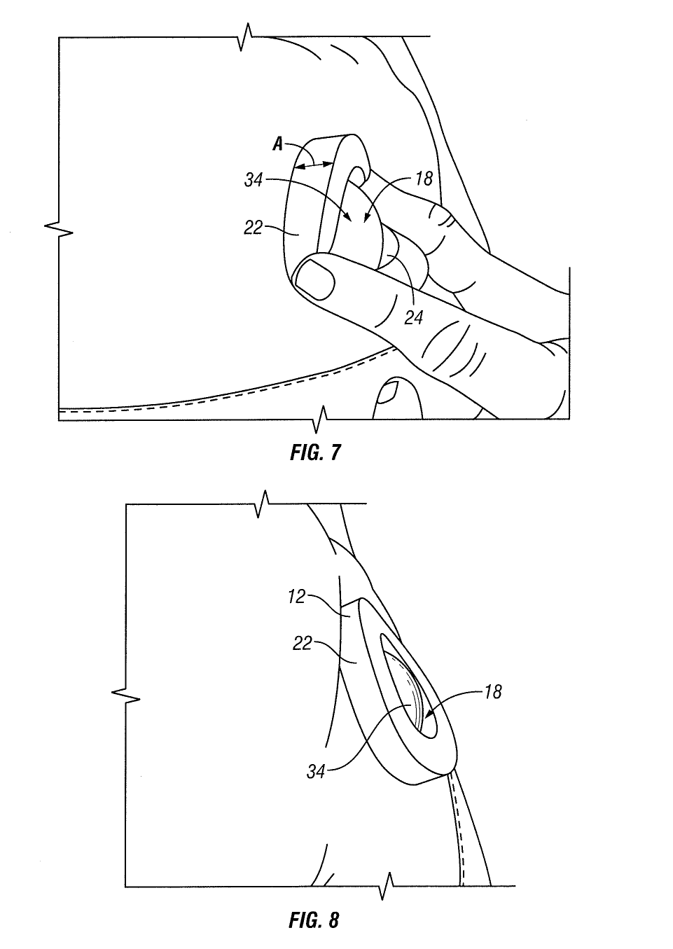

FIGS. 6 and 7 show the application of the protective device 12 to the skin of a user. In the example shown, the front face 26 or rear face 28 with the adhesive 32 exposed is adhered to the user's skin proximate the implanted medical device by applying pressure on the protective device 12 toward the user's body. This provides a cushion to protect the tender area 34 near the implanted medical device. The tender area 34 of the user's body in which skin protrudes outwardly due to an implanted medical device will be referred to as the “protruding area.” In the example shown, the protective device 12 has a thickness “A.” In one embodiment, the thickness “A” is dimensioned in a manner to reduce the chance that direct contact will be made with the protruding area, which would cause discomfort to the user. For example, the thickness “A” may be greater than the distance that the skin protrudes due to the implanted medical device. In the example shown, the opening 18 has a diameter “B.” In one embodiment, the opening 18 is dimensioned to surround the implanted medical device sufficient to cushion the protruding area so that discomfort to the user is minimized.

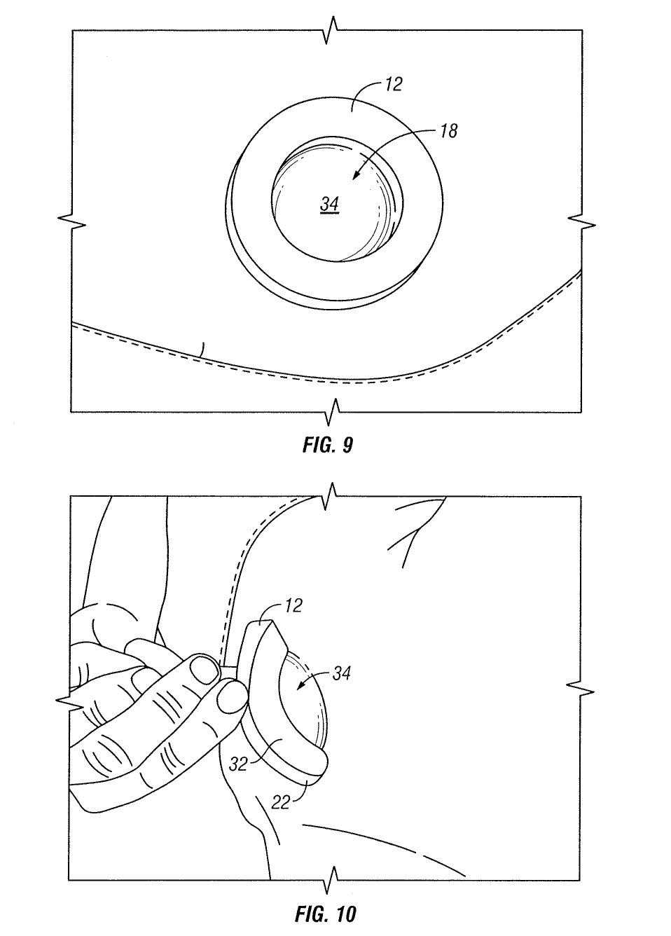

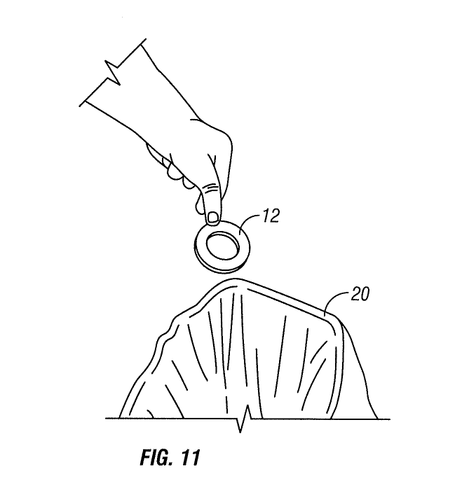

FIGS. 8 and 9 reflect the protective device adhered to the user from both front and side views. FIGS. 10 and 11 show the removal and disposal of the protective device 12 by the user once the use is complete.

For purposes of example only, the protective device may be approximately 2.75″ wide, 0.375″ thick with a hole of 1.32″ in diameter cut in the middle. In one embodiment, a double-sided adhesive could be used with the protective device. The double-sided adhesive would typically be applied to one side of the non-latex foam material. The other side of the adhesive would be covered by a protective film and remain intact until the protective device has been removed from the excess foam packaging material by the user. The protective film is removed from the adhesive on the protective device and then is applied to the skin area surrounding the implanted medical device that is protruding under the skin. This allows for a strong bond between the protective device and the body, allowing objects to contact and otherwise rub against the protective device instead of the protruding skin area without the device coming off the body. The protective device's thickness is conformed to keep seatbelts, bra straps, and other types of clothing from irritating the protruding skin caused by an implanted medical device, thus providing the user with increased comfort.

Due to the type of adhesive, the same protective device may be used repeatedly, such as for several days by the user. For example, the protective device may be removed prior to and during chemotherapy or other type of infusion treatment and reapplied after each treatment. The protective device will not interfere with the implanted medical device or access thereto by medical professionals.

Embodiments are contemplated in which the protective device may not necessarily be annular-shaped. In some embodiments, the adhesive may be integrally formed on the protective device or applied at the time of attachment to the body. Embodiments are also contemplated in which foam or other cushioning materials may be used for the protective device.

Although the present disclosure has been described with reference to particular means, materials and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. A protective device for protecting a tender area proximate an implanted medical device, the device comprising:
a cushion layer with an opening dimensioned to receive at least a portion of a protruding area of skin that protrudes outwardly due to an implanted medical device, wherein the cushion layer formed from a material configured to dampen force applied to the protruding area, wherein the cushion layer defines a first face of the protective device, wherein the cushion layer has a thickness that is greater than a distance that the protruding area extends outwardly due to the implanted medical device;
an adhesive layer with an opening corresponding with the opening in the cushion layer for attaching the cushion layer to the skin, wherein the adhesive layer defines a second face of the protective device that is on an opposite side of the protective device from the first face; and
a protective film covering the adhesive layer.

2. The device of claim 1, wherein the protruding area of skin extends into the opening in the cushion layer when the adhesive layer is attached to the skin.

3. The device of claim 1, wherein the cushion layer is configured to transmit a force impacting the cushion layer to an area surrounding the protruding area.

4. The device of claim 1, wherein the cushion layer has a ring-like shape.

5. The device of claim 4, wherein the opening is approximately concentric with respect to the cushion layer.

6. The device of claim 1, wherein the cushion layer is formed from foam.

7. The device of claim 1, wherein the foam is of the type that will absorb at least a portion of force that would otherwise be applied to the protruding area.

8. A method for protecting an implanted medical device, the method comprising the steps of:
providing an implanted medical device subcutaneously mounted in a human such that an area of skin protrudes outwardly due to the implanted medical device;
adhering a protective device to skin surrounding the implanted medical device,
wherein the protective device includes a first face with an annular cushion layer defining an opening dimensioned to receive the area of skin protruding outwardly due to the implanted medical device, wherein the cushion layer has a thickness that is greater than a distance that the skin protrudes due to the implanted medical device.; and
wherein the protective device includes an adhesive layer for attaching the cushion layer to the skin, wherein the adhesive layer is on a face of the protective device opposite the cushion layer.

9. The device of claim 8, wherein the cushion layer is configured to surround an area of skin that protrudes due to the implanted medical device.

10. The device of claim 8, wherein the cushion layer is configured to transmit a force impacting the cushion layer to an area surrounding the area protruding due to the implanted medical device, but minimize the force impacting the protruding area.

11. The device of claim 8, further comprising a protective film covering the adhesive layer.

12. The device of claim 8, wherein the cushion layer has a ring-like shape.

13. The device of claim 12, wherein the opening and the cushion layer are approximately concentric.

14. The device of claim 8, wherein the cushion layer is formed from foam.

15. A protective device for protecting a tender area proximate an implanted medical device, the device consisting of:
a cushion layer with an opening dimensioned to receive at least a portion of a protruding area of skin that protrudes outwardly due to an implanted medical device, wherein the cushion layer formed from a material configured to dampen force applied to the protruding area, wherein the cushion layer defines a first face of the protective device, wherein the cushion layer has a thickness that is greater than a distance that the protruding area extends outwardly due to the implanted medical device;
an adhesive layer with an opening corresponding with the opening in the cushion layer for attaching the cushion layer to the skin, wherein the adhesive layer defines a second face of the protective device that is on an opposite side of the protective device from the first face; and
a protective film covering the adhesive layer.

16. The device of claim 15, wherein the opening is approximately concentric with respect to the cushion layer.

17. The device of claim 15, wherein the cushion layer has a substantially circular shape.

18. The device of claim 17, wherein the cushion layer is a non-latex foam.
